(19) United States
(12) Patent Application Publication
(10) Pub. No.: US 2024/0423758 A1
(43) Pub. Date: Dec. 26, 2024

(54) METHOD OF ADMINISTERING ADHESIVE TO BOND ORTHODONTIC BRACKETS

(71) Applicant: Swift Health Systems Inc., Irvine, CA (US)

(72) Inventors: Hongsheng Tong, Yorba Linda, CA (US); Jichele Sanders, Chantilly, VA (US); Shermaine Dayot, Irvine, CA (US); Ishan Shinde, Santa Ana, CA (US)

(21) Appl. No.: 18/777,292

(22) Filed: Jul. 18, 2024

Related U.S. Application Data
(62) Division of application No. 17/690,770, filed on Mar. 9, 2022, now Pat. No. 12,053,345.
(60) Provisional application No. 63/240,627, filed on Sep. 3, 2021.

(51) Int. Cl. A61C 7/02; A61B 5/00; A61C 7/00; A61C 7/14; A61C 7/16

(52) U.S. Cl. CPC A61C 7/02; A61B 5/0077; A61C 7/002; A61C 7/146; A61C 7/16; A61C 2202/01

(57) ABSTRACT

Methods of administering a quantity of adhesive to bond orthodontic brackets is disclosed herein. The quantity of adhesive to bond a selected bracket to a tooth at a specific location thereon can be calculated based on a known geometry of the selected bracket and a digital model of the patient's teeth. An instruction guide can visually indicate with a graphic the amount of adhesive to dispense from a syringe or other device to bond the selected bracket to the surface of the tooth at a specific location planned in the digital model.

### CROSS REFERENCE TO RELATED APPLICATIONS

This application is a divisional of U.S. application Ser. No. 17/690,770, filed Mar. 9, 2022, which claims priority to U.S. Provisional Patent Application No. 63/240,627, filed Sep. 3, 2021, each of which are incorporated herein by reference in their entireties. Any and all applications, if any, for which a foreign or domestic priority claim is identified in the Application Data Sheet of the present application is hereby incorporated by reference under 37 CFR 1.57.

### BACKGROUND

### Field

The present disclosure relates in some aspects to methods of administering adhesive to bond orthodontic brackets to a patient's teeth.

### SUMMARY

It can be difficult to determine a quantity of adhesive that is suitable for bonding an orthodontic bracket to a patient's tooth. If too much adhesive is used, a clinician may need to clean up excess adhesive post bonding, which can waste adhesive and extend the length of a patient's chair time. If too little adhesive is used, a weak bond may be formed between the orthodontic bracket and the patient's tooth, which may result in the orthodontic bracket inadvertently debonding from the tooth. Accordingly, methods, systems, and apparatuses are disclosed herein which determine and/or administer a quantity of adhesive for bonding an orthotic bracket to a patient's tooth.

In some variants, a method of determining a quantity of adhesive to bond an orthodontic bracket to a tooth surface is disclosed herein. The method can include scanning an inside of a mouth of a patient. The method can include creating a digital model of teeth of the patient. The method can include selecting a digital bracket to be placed on a digital tooth of the digital model. The digital bracket having a bonding surface with a known geometry. The method can include positioning the digital bracket at a location on a surface of the digital tooth of the digital model. The method can include determining a quantity of adhesive to bond a bonding surface of a physical orthodontic bracket, corresponding to the bonding surface of the digital bracket, to a location of a surface of a physical tooth, corresponding to the location of the surface of the digital tooth, based at least partially on the known geometry of the bonding surface of the digital bracket and a geometry at the location of the surface of the digital tooth where the digital bracket is positioned.

In some variants, scanning the inside of the mouth of the patient is performed using a mobile device.

In some variants, scanning the inside of the mouth of the patient can include 3D scanning.

In some variants, scanning the inside of the mouth of the patient can include taking 2D images.

In some variants, selecting the digital bracket can include selecting the digital bracket from a plurality of digital brackets having unique characteristics.

In some variants, the digital bracket can be automatically selected based on one or more characteristics of the digital tooth.

In some variants, selecting the digital bracket can include selecting the digital bracket from a plurality of suggested digital brackets.

In some variants, the digital bonding surface and corresponding physical bonding surface can include one or more contours to facilitate improved bonding.

In some variants, the quantity of adhesive to bond the bonding surface of the physical orthodontic bracket to the location of the surface of the physical tooth can be determined by an algorithm.

In some variants, a method of determining a quantity of adhesive to be applied to an orthodontic bracket for bonding is disclosed herein. The method can include dispensing an adhesive from a container. The method can include visually comparing a size of a profile of the dispensed adhesive with an adhesive quantity graphic on an instructional guide. The method can include discontinuing the dispensing of the adhesive from the container when the profile of the dispensed adhesive matches the adhesive quantity graphic on the instructional guide.

In some variants, the method can include transferring the dispensed adhesive to a bonding surface of an orthodontic bracket.

In some variants, the method can include distributing the dispensed adhesive over the contact surface of the orthodontic bracket.

In some variants, distributing the dispensed adhesive over the contact surface of the orthodontic bracket can include using a microbrush.

In some variants, the method can include loading a well of an indirect bonding tray (IDB) tray with the orthodontic bracket.

In some variants, the method can include storing the loaded IDB tray in a light-proof case to prevent the adhesive from curing.

In some variants, the method can include placing the IDB tray on the teeth of the patient to position the orthodontic bracket at a preplanned position on a tooth.

In some variants, the method can include exposing the adhesive to UV light to cure the adhesive.

In some variants, a width of an opening of the container and a width of the adhesive quantity graphic can be the same such that visually comparing the size of the profile of the dispensed adhesive with the adhesive quantity graphic on the instructional guide includes comparing a length of the profile of the dispensed adhesive with a length of the adhesive quantity graphic.

In some variants, the container can be a syringe.

In some variants, the method can include cutting the dispensed adhesive from an end of the container.

In some variants, cutting the dispensed adhesive from the end of the container can include cutting with a composite instrument. The composite instrument can be thin.

In some variants, a kit is disclosed herein. The kit can include a container that can have a cavity that can to hold an adhesive therein. The kit can include an instructional guide that can include an adhesive quantity graphic. The adhesive quantity graphic can be visually compared with a profile of adhesive dispensed from the container to determine a quantity of dispensed adhesive to be applied to an orthodontic bracket for bonding on a location of a tooth of a patient.

In some variants, the container can be a syringe.

In some variants, the cavity can hold the adhesive therein.

In some variants, the kit can include a second container that can house the adhesive to be transferred to the cavity of the container for dispensing.

In some variants, the container can include an opening through which adhesive housed in the cavity can be dispensed.

In some variants, the opening of the container can have a width that is the same as a width of the adhesive quantity graphic such that a length of the profile of adhesive dispensed from the container can be visually compared with a length of the adhesive quantity graphic to determine the quantity of dispensed adhesive to be applied to the orthodontic bracket for bonding.

In some variants, the instructional guide can include a plurality of adhesive quantity graphics and a plurality of tooth identifiers corresponding to respective teeth of a patient. Each of the plurality of adhesive quantity graphics can be, respectively, juxtaposed one of the plurality of tooth identifiers to visually indicate the quantity of dispensed adhesive to be applied to the orthodontic bracket corresponding to the tooth identifier.

In some variants, the kit can include an instrument to cut the dispensed adhesive from the container.

In some variants, the instrument is a composite instrument. The composite instrument can be thin.

In some variants, the kit can include a microbrush to distribute the dispensed adhesive over a contact surface of the orthodontic bracket.

In some variants, the kit can include an indirect bonding tray (IDB) tray.

In some variants, the kit can include a light-proof case that can to delay or prevent curing of the adhesive.

In some variants, the instructional guide can include patient identifying information.

In some variants, the kit can include adhesive.

In some variants, the kit can include one or more archforms.

In some variants, the one or more archforms can be made of a shape memory material and set in a custom nonplanar shape that can move teeth of a patient.

In some variants, the can include a plurality of orthodontic brackets.

In some variants, the plurality of orthodontic brackets can be loaded in an indirect bonding (IDB) tray.

In some variants, the adhesive quantity graphic can be printed on the instructional guide.

### BRIEF DESCRIPTION OF THE DRAWINGS

These drawings are illustrative embodiments and do not present all possible embodiments of this invention. The illustrated embodiments are intended to illustrate, but not to limit, the scope of protection. Various features of the different disclosed embodiments can be combined to form further embodiments, which are part of this disclosure.

FIG. 1 illustrates a patient's teeth, including the upper and lower dental arches.

FIGS. 2A and 2B illustrate views of an orthodontic bracket.

FIGS. 3A and 3B illustrate views of another orthodontic bracket.

FIG. 4 illustrates an archform.

FIG. 5 illustrates the bracket of FIGS. 2A and 2B with a bracket connector of an archform coupled to the bracket.

FIG. 6 illustrates the bracket of FIGS. 3A and 3B bonded to the surface of a tooth and receiving a bracket connector of an archform.

FIG. 7 illustrates a method of determining a quantity of adhesive to bond an orthodontic bracket to a tooth surface.

FIG. 8 illustrates an instruction guide that graphically represents at scale a quantity of adhesive to bond a selected bracket to a specific tooth.

FIG. 9 illustrates an indirect bonding tray.

FIG. 10 illustrates a method of applying a quantity of adhesive to orthodontic brackets for bonding.

### DETAILED DESCRIPTION

Malocclusion of the teeth may be treated using orthodontic brackets and archforms to move the patient's teeth using non-sliding mechanics. For example, scans of a patient's teeth can be taken and a digital model of the patient's teeth can be created, at least in part, from the scans. The teeth of the digital model can be moved from positions of malocclusion (e.g., first positions) to second positions, which may be a final expected alignment of the teeth. Digital brackets can be placed, respectively, on the lingual or buccal surfaces of the teeth in the digital model. In some variants, the digital brackets can be placed before moving the teeth of the digital model from the positions of malocclusion (e.g., first positions) to the second positions.

A physical fixture can be created (e.g., 3D printed, machined, and/or otherwise formed) based on the digital model with the teeth in the second positions and the digital brackets placed. The physical fixture can include retention features (e.g., hooks, slots, locks, holders, etc.) that can retain portions of an archform such as bracket connectors. The retention features can be positioned based on the corresponding positioning of the digital brackets in the digital model with the teeth in the second positions, such that the relative positioning of the retention features to each other is the same as or similar to the relative positioning of the digital brackets to each other in the digital model. In some variants, the physical fixture can be a physical model of the patient's teeth corresponding to the digital model of the patient's teeth in the second positions and the retention features positioned on the teeth of the physical model can be based on the corresponding positioning of the digital brackets in the digital model.

An archform, which may also be referred to as an archwire, can be made of a shape memory material, such as nickel-titanium alloy (e.g., Nitinol). The archform can be cut (e.g., laser, waterjet, etc.) from a sheet of material (e.g., shape memory material). The archform can include bracket connectors that can be coupled to brackets and interproximal segments, such as interproximal loops, that can be configured to move one or more teeth of the patient. When cut from the sheet of material, the archform can have a substantially flat two-dimensional shape. The archform can be deflected and coupled to the physical fixture to assume a custom nonplanar shape. Specifically, the bracket connectors of the archform can be coupled to the retention features of the physical fixture. While retained in the custom nonplanar shape, the archform can be set, which can accomplished via exposure to heat. Setting the archform can set a new default or memorized position for the archform such that the archform is biased toward the memorized custom nonplanar shape when deflected therefrom. Accordingly, if the archform is deflected from the memorized custom nonplanar shape, the archform can exert forces to return the archform back toward the memorized custom nonplanar shape.

An indirect bonding (IDB) tray can be formed based on the digital model. The teeth of the digital model, with the digital brackets disposed thereon, can be returned back to the positions of malocclusion that can reflect the current positions of the patient's teeth. In some variants, the IDB tray can be formed based on the digital model with digital brackets placed on the maloccluded teeth of the digital model prior to movement of the teeth to the second positions. An IDB tray can be 3D printed based on the digital model and/or over molded on a physical model of the patient's teeth. The IDB tray can be sized and shaped to fit over the teeth of the patient. The IDB tray can include wells (e.g., pockets, recesses, etc.) that can house orthodontic brackets therein. The wells can be positioned based on the corresponding positioning of the digital brackets in the digital model.

Orthodontic brackets can be placed in respective wells of the IDB tray with contact surfaces (e.g., bonding surfaces) exposed. An adhesive can be applied to the contact surfaces and the loaded IDB tray can be placed over the teeth of the patient, positioning the orthodontic brackets at locations on the teeth of the patient that correspond to the positioning of the digital brackets on the teeth in the digital model. The orthodontic brackets can bond to the teeth of the patient, which can be facilitated by exposing the adhesive to light (e.g., UV light), heat, low temperatures, and/or chemical(s).

As described herein, it can be difficult to determine a quantity of adhesive that is suitable for bonding an orthodontic bracket to a patient's tooth. If too much adhesive is used, a clinician may need to clean up excess adhesive post bonding, which can waste adhesive and extend the length of a patient's chair time. If too little adhesive is used, a weak bond may be formed between the orthodontic bracket and the patient's tooth, which may result in the orthodontic bracket inadvertently debonding from the tooth.

With the orthodontic brackets bonded to the teeth of the patient, the archform can be deflected from the memorized custom nonplanar shape and coupled to the bonded orthodontic brackets. The bracket connectors of the archform can be coupled, e.g., locked, to the bonded orthodontic brackets such that the archform does not slide with respect to the brackets. As described, the deflected archform can exert forces on the teeth of the patient as the archform exerts forces to move toward the undeflected position (e.g., memorized custom nonplanar shape), which can move the teeth of the patient, using non-sliding mechanics, toward seconds positions that correspond to the second positions of the teeth in the digital model (e.g., final alignment of the teeth).

In some variants, a series of archforms can be sequentially installed in the patient's mouth (e.g., coupled to the brackets) and then replaced to move the patient's teeth from positions of malocclusion to second positions. For example, an initial archform set in the custom nonplanar shape may be used for an initial stage of treatment for initially moving the teeth of the patient toward the second positions. An intermediate archform set in the custom nonplanar shape, which may be stiffer than the initial archform, may be used for an intermediate stage of treatment for moving the teeth of the patient closer toward the second positions. A final archform set in the custom nonplanar shape, which may be stiffer than the intermediate archform, may be used for a final stage of treatment for moving the teeth of the patient closer toward or to the second positions.

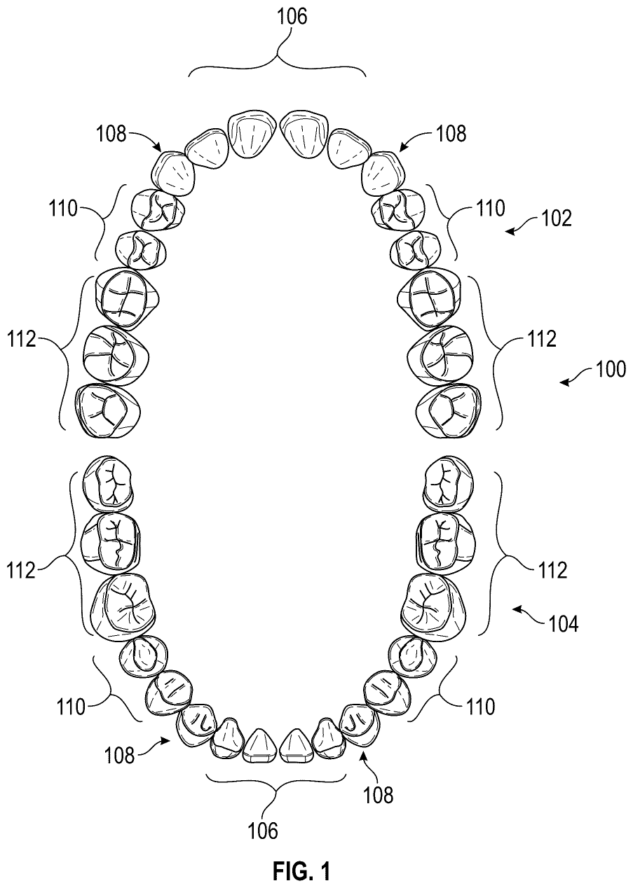

FIG. 1 illustrates teeth 100 of a patient, including the upper dental arch 102 and the lower dental arch 104. The human mouth includes a variety of teeth, including incisors 106, canines 108, premolars 110, and molars 112. Each of the foregoing varieties can have different characteristics, sizes, shapes, contours, and/or purposes. For example, incisors 106 are typically smaller than molars and are used to bit into and tear food, while molars 112 include large and relatively flat biting surfaces to crush and grind food. Furthermore, the same variety of teeth can vary depending on whether positioned on the upper dental arch 102 or lower dental arch 104. For example, the upper incisors 106 are typically larger than lower incisors 106. Furthermore, the same tooth can have different characteristics, sizes, shapes, contours, and/or purposes from person to person. Accordingly, in some instances, a variety of brackets with different characteristics, such as size, may be bonded to the surfaces of the teeth of a patient to accommodate different teeth within the patient's mouth.

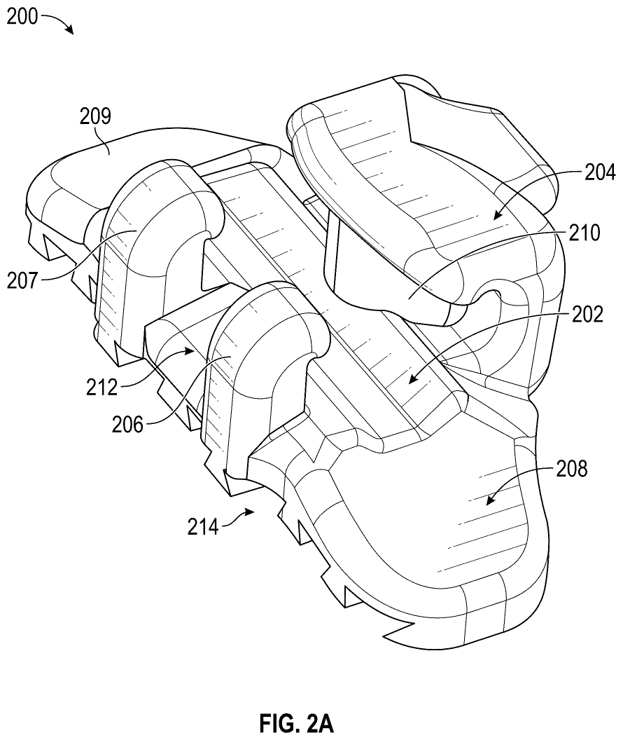

For example, FIGS. 2A and 2B illustrate a bracket 200, which may be at least used on molars 112. As shown, the bracket 200 can include lateral extensions or wings 208, 209 that extend in the medial-distal direction when the bracket 200 is bonded to a tooth. The relatively larger size of molars 112 may permit the incorporation of lateral extensions 208, 209. The lateral extensions 208, 209 can facilitate increased control (e.g., rotational) of a molar 112. The bracket 200 can include a contact or bonding surface 214 that is configured to be bonded to the surface of a tooth of the patient. The contact surface 214 can include contours or grooves 215, which can increase surface area of the contact surface 214 to improve bonding between the bracket 200 and the surface of the tooth with adhesive. As illustrated, the lateral extensions 208, 209 increase the size of the contact surface 214, which can further increase the strength of the bond between the bracket 200 and the surface of the tooth.

The bracket 200 can include a variety of features that facilitate coupling to an archform. The bracket 200 can include a slot 202, which can be disposed between a retainer 204 and stops 206, 207. The slot 202 can receive a bracket connector of an archform therein such that the archform does not slide with respect to the bracket 200. The retainer 204 and the stops 206, 207 can at least prevent movement of the bracket connector relative to the bracket 200 in the gingival-occlusal direction. The bracket 200 can include a spring 210 (e.g., C-spring) that can lock the bracket connector within the slot 202. The spring 210 can be disposed in the retainer 204 and push the bracket connector against the stops 206, 207 to lock the bracket connector within the slot 202. A gap 212 can space apart the stops 206, 207 and receive a portion of the bracket connector therein such that the stops 206, 207 impede medial-distal movement of the bracket connector relative to the bracket 200.

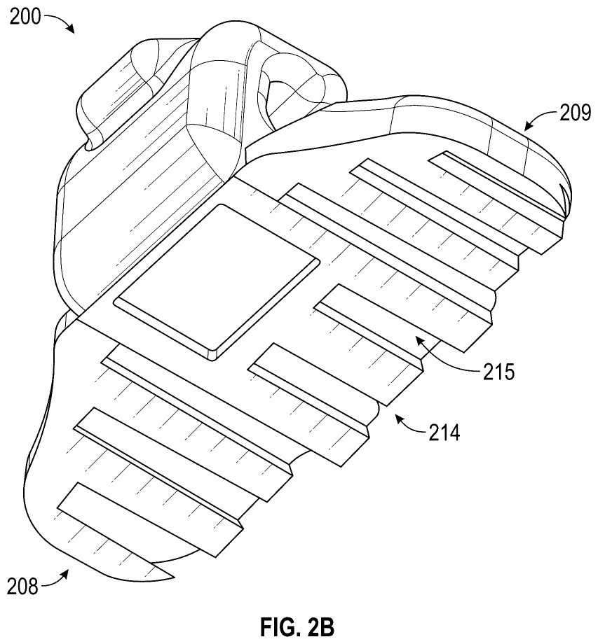

FIGS. 3A and 3B illustrate a bracket 300, which may be used at least on premolars 110, canines 108, and/or incisors 106. As shown, the bracket 300 omits the lateral extensions illustrated in FIGS. 2A and 2B, which can at least be attributed to the smaller size of the premolars 110, canines 108, and/or incisors 106 compared to the molars 112. Without the lateral extensions, the contact or bonding surface 214 of the bracket 300 is smaller than the contact or bonding surface 214 of the bracket 200. Additionally, the contours 215 in the contact surface 214 of the bracket 300 may be different than the those of the bracket 200. At least these differences in bracket size and characteristics can change the quantity of adhesive used to bond the brackets to the surface of the teeth. For example, a quantity of adhesive used to bond the bracket 200 to a molar 112 may be larger than a quantity of adhesive used to bond the bracket 300 to a premolar 110, canine 108, and/or incisor 106.

The bracket 300 can include similar features to retain a portion of the archform (e.g., the bracket connector). The bracket 300 can at least include a slot 202, retainer 204, stops 206, 207, gap 212 disposed between the stops 206, 207, and/or spring 210 as described in reference to the bracket 200.

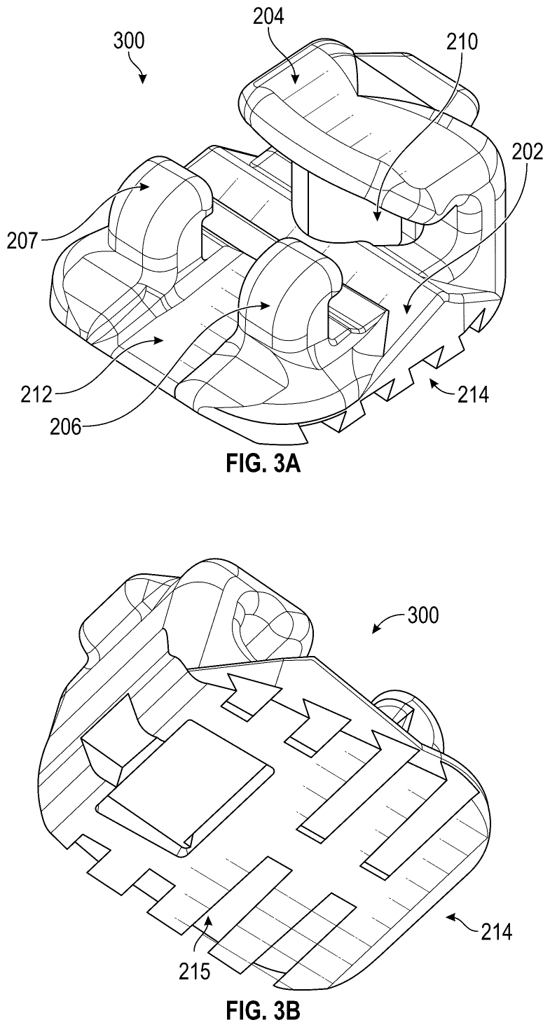

FIG. 4 illustrates an archform 400, which can also be referred to as an archwire. The archform 400 can have a rectangular cross-section. The archform 400, as described herein, can be cut from a sheet of material, such as shape memory material (e.g., nickel titanium). The archform 400 can include a plurality of bracket connectors 404 that can be coupled to orthodontic brackets to install the archform 400 in the mouth of a patient. The archform 400 can include a plurality of interproximal segments 402. The interproximal segments 402 can be disposed between adjacent bracket connectors 404. The interproximal segment 402 can include loops. The loops can extend in a gingival direction when the archform 400 is installed in the mouth, which can improve aesthetics and/or facilitate flossing. The loops can open to move adjacent teeth apart from each other. The loops can close to move adjacent teeth closer together.

As shown, the archform 400 is in a two-dimensional shape. As described herein, the archform 400 can be set in a custom nonplanar shape using a fixture based on a digital model of a patient's teeth in second positions, which may be a final alignment of the teeth. The archform 400 can be held in the custom nonplanar shape by the fixture and set by exposure to heat such that the custom nonplanar shape is the default or memorized position of the archform 400. The archform 400 can follow the entire upper or lower dental arch of a patient or a segment thereof.

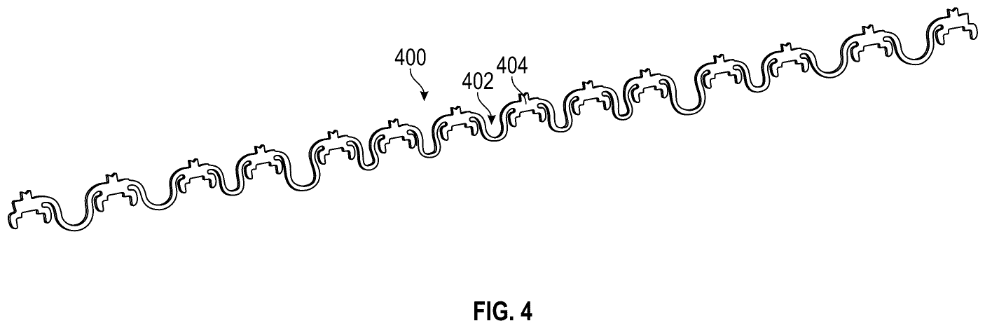

FIG. 5 illustrates the bracket connector 404 coupled with the bracket 200 such that the bracket connector 404 will not slide with respect to the bracket 200. The bracket connector 404 is disposed within the slot 202 of the bracket 200. The stops 206, 207 and retainer 204 cooperate to retain the bracket connector 404 within the slot and prevent movement of the bracket connector 404 in the occlusal-gingival direction. A portion of the bracket connector 404, e.g., the tongue or tab, can be disposed in the gap 212 between the stops 206, 207 which can help impede the bracket connector 404 from sliding relative to the bracket 200 in the medial-distal direction. As described herein, the spring 210 can push the bracket connector 404 against the stops 206, 207 locking the bracket connector 404 within the bracket 200. In some variants, the bracket connector 404 may be tied to the bracket 200.

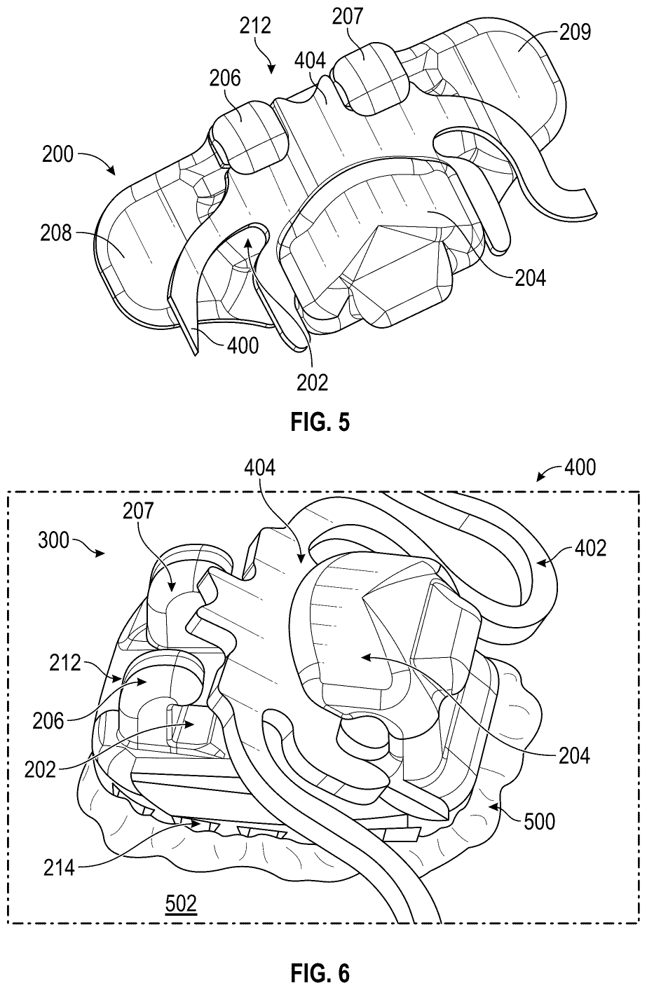

FIG. 6 shows the bracket 300 bonded to a tooth surface 502 with a bonding agent or adhesive 500. The adhesive 500 can, in some variants, cure from exposure to air. In some variants, the adhesive 500 can cure from exposure to light, such as UV light. In some variants, the bracket 300 with adhesive applied to the contact surface 214 can be disposed on the tooth surface 502 and, after placement, the adhesive 500 can be exposed to UV light until the adhesive 500 is cured or substantially cured. As shown, the adhesive 500 is extending beyond a periphery of the contact surface 214 which, in some instances, may be removed post bonding. In some variants, an amount of adhesive 500 can be applied to the contact surface 214 for bonding such that the adhesive 500 does not substantially extend beyond a periphery of the contact surface 214 after placement on the tooth surface 502. As shown, the bracket connector 404 is being installed into the slot 202 of the bracket 300.

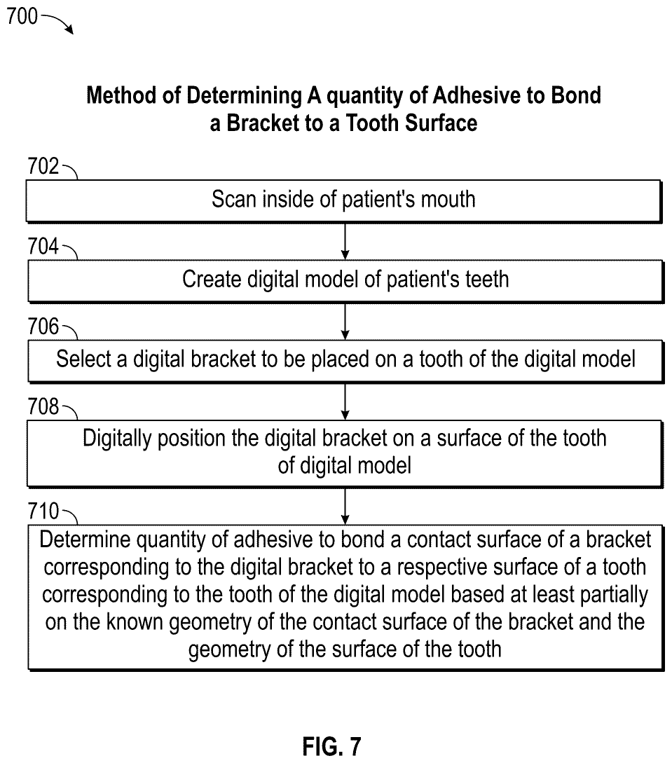

FIG. 7 illustrates an example method 700 of determining a quantity of adhesive to bond a bracket to a tooth surface. This flow diagram is provided for the purpose of facilitating description of aspects of some embodiments. The diagram does not attempt to illustrate all aspects of the disclosure and should not be considered limiting.

At block 702, a scan (e.g., 3D scans and/or 2D scans) can be taken of the inside of the patient's mouth (e.g., dental arches). The scan can capture data regarding the type, size, shape, contours, surface features, and/or other characteristics of the patient's teeth. The scans can be taken by the patient, caretaker of the patient, and/or clinician. The scan can be performed using a camera and/or sensor of a computer, device connected to a computer, and/or a mobile device, such as a smartphone. In some variants, an application can be used to perform the scans-providing the patient with instructions on how to perform the scan and when a scan is successful. The scan can be performed using the mobile device's built-in camera or via an attachment that operatively connects to the mobile device or computer. In some variants, the scan can be captured using a digital intra-oral scanner and/or a cone-beam computed tomography (CBCT) X-ray scanner. The scanned data can be sent to a manufacturer of orthodontic appliances for processing and/or use.

At block 704, a digital model of the patient's teeth in first positions can be created based on scans of the inside of the patient's mouth. The first positions can correspond to the current positions of the patient's teeth which can be maloccluded positions. The digital model can represent the unique size, shape, contours, surface features, and/or other characteristics of the patient's teeth. The digital model can be displayed to an operator for viewing and/or manipulation. In some variants, the digital model can be automatically generated by software implemented on a computing device using the scans of the inside of the patient's mouth.

At block 706, a digital bracket suitable for bonding on a surface of a tooth can be selected from a variety of digital brackets. As discussed herein, certain types of brackets may be more suitable and/or preferred for bonding on a given tooth but not others. For example, a bracket with lateral extensions may be suitable for bonding on the molars but not on the lower incisors 106 because of differences in size and shape. In some variants, an operator can select a bracket from a variety of brackets for placement on a tooth based on the type of tooth and/or features of the tooth shown in the digital model. In some variants, software implemented on a computing device may suggest one or more brackets from a variety of brackets for placement on a tooth based on the type of tooth and/or features of the relevant tooth. In some variants, software implemented on a computing device may select a bracket from a variety of brackets based on the type of tooth and/or features of the relevant tooth.

At block 708, the selected digital bracket can be digitally placed on the surface of the relevant tooth of the digital model. In some variants, the operator can place the selected digital bracket on the surface (e.g., lingual or buccal surface) of the tooth in the digital model. In some variants, the selected digital bracket can be automatically placed on the surface of the tooth in the digital model. The processes described in reference to block 706 and block 708 can be repeated to select and respectively place a plurality of digital orthodontic brackets on surfaces of the teeth in the digital model, which can include every tooth of the digital model or only some of the teeth of the digital model.

At block 710, a quantity (e.g., volume) of adhesive can be determined to bond a contact surface of a bracket corresponding to the selected digital bracket to a respective surface of a tooth of the patient corresponding to the tooth of the digital model. The determination of the quantity of adhesive can at least be based on the known geometry of the contact surface of the bracket and the geometry of the respective surface of the tooth (using data regarding the type, size, shape, contours, surface features, and/or other characteristics from the scan and/or digital model of the patient's teeth) at the location the bracket will be placed, which can be based on the digital model. As described herein, a variety of orthodontic brackets can be used that have contact surfaces of varying geometry, e.g., sizes, shapes, etc., which can be known by a computing device having one or more processors, memory, instructions, a communication interface, a display, controllers, a user interface, and/or other features requisite to determine the quantity of adhesive to perform a bond. As more orthodontic brackets are developed, data indicative of the geometry of the bracket, including the contact surface, can be stored in the memory of the computing device. In some variants, new orthodontic brackets can be scanned, and the scanned data, indicative of the geometry of the bracket, including the contact surface, can be stored in the memory of the computing device. As described herein, based on the known geometry of the bracket and the scan data and/or digital model of the patient's teeth, the computing device can determine a quantity of adhesive to bond the selected orthodontic bracket at a specific location on the patient's teeth using an algorithm. The quantity of adhesive can change based on the type of bracket selected, type of tooth that the bracket will be bonded to, the unique characteristics of the tooth that the bracket will be bonded to, the location on the tooth that the bracket will be bonded to, the type of adhesive, and/or others.

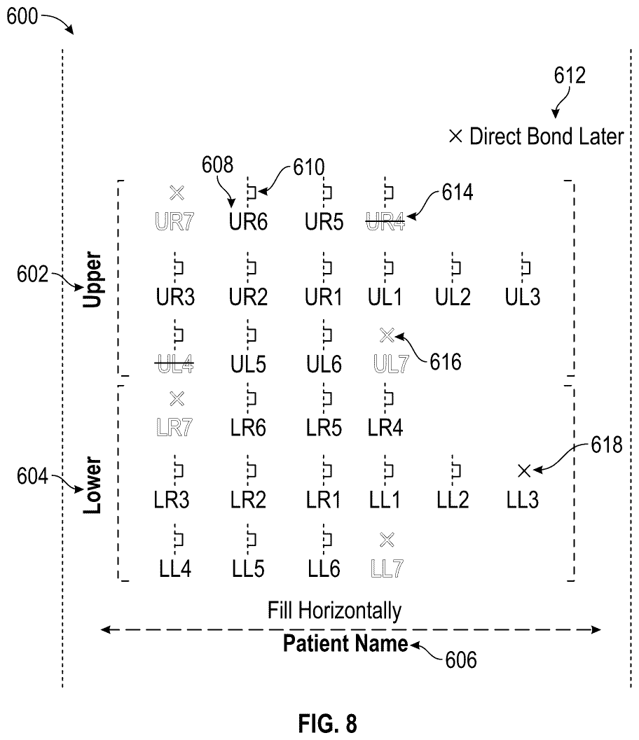

FIG. 8 illustrates an instruction guide 600 (e.g., graphic, instruction card, etc.) that can be used to instruct a clinician or operator as to the quantity (e.g., volume) of adhesive to apply to a contact surface of a orthodontic bracket to bond a specific bracket on a specific tooth at a specific location. In some variants, the instruction guide 600 can be printed onto a sheet of material, such as paper. In some variants, the instruction guide 600 can be included in a kit that is specific to a patient. The instruction guide 600 can include a patient identifier 606 to pair the instruction guide 600 with the patient. The kit can include a plurality of orthodontic brackets, an IDB tray, one or more archforms, an installation tool, adhesive, primer, and/or other components or features described herein. In some variants, the instruction guide 600 can be displayed on a screen. The instruction guide 600 can include at least two sections for organization, which can include the upper section 602 that corresponds to the teeth of the upper arch of a patient and the lower section 604 that corresponds to the teeth of the lower arch of the patient. All or some of the teeth of the patient can respectively correspond to a tooth identifier 608 (e.g., UR6, UR5, LR5, LR1, LL1, etc.) on the instruction guide 600 that can be positioned in the upper section 602 or lower section 604.

An adhesive quantity graphic 610 can be juxtaposed a corresponding tooth identifier 608. The adhesive quantity graphic 610 can visually indicate, at scale, a quantity (e.g., volume) of adhesive, which can be determined based on method 700, that should be applied to a corresponding orthodontic bracket to be used to bond the bracket at a specific location on a surface of the tooth corresponding to the tooth identifier 608. The adhesive can be stored in a syringe, container, squeeze container, tube, squeeze tube, and/or other device with a dispenser opening of a known size such that the volume of adhesive dispensed from the syringe is a product of the size of the dispenser opening multiplied by the length of the dispensed adhesive. The width of the adhesive quantity graphic 610 can be the same as the width (e.g., diameter) of the opening of the syringe or other device. Accordingly, a computing device can determine a quantity (e.g., volume) of adhesive to bond the selected bracket to the surface of the tooth at a specific location on the tooth, as described herein, and create an adhesive quantity graphic 610 with a width that is the size of the opening of the syringe or other device and a sufficient length to correlate to the determined volume of adhesive to bond the selected bracket to the surface of the tooth at the specific location on the tooth as planned in the digital model. In practice, a clinician can dispense adhesive from the syringe through the dispensing opening until a length (e.g., size) of a side or horizontal profile of the dispensed adhesive visually matches the length of the adhesive quantity graphic 610 to ensure that a suitable amount of adhesive is applied to the contact surface of the selected orthodontic bracket for bonding.

The instruction guide 600 can include a legend 612 that indicates the meaning of certain graphics of the instruction guide 600. The instruction guide 600 can include a direct bond later indicator 618, such as an X mark in an eye-catching color such as green or others, juxtaposed a tooth identifier 608 to indicate that the selected bracket for the corresponding tooth should be direct bonded later rather than bonded up with the use of an IDB tray, as described herein. The instruction guide 600 can include a do-not-treat indicator 616, which can indicate that a tooth is not being treated (e.g., no bracket should be bonded to the tooth). The do-not-bond indicator 616 can include a tooth identifier, which can be greyed out, and a X mark, which can be greyed out. The instruction guide 600 can include struck through tooth identifiers 614, which can indicate that the patient is missing a tooth at that location. The struck through tooth identifiers 614 can be greyed out.

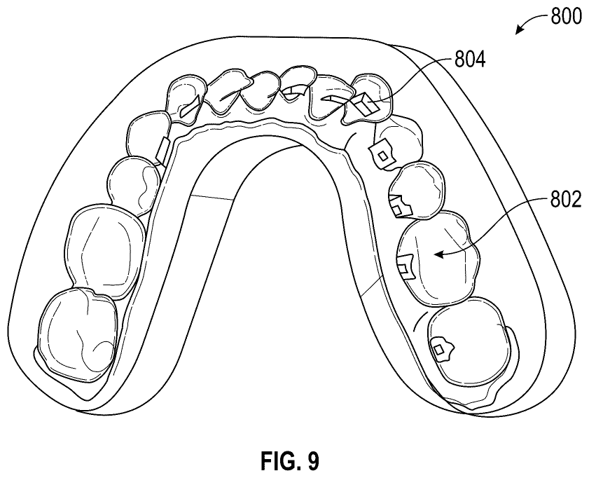

FIG. 9 illustrates an indirect bonding tray (IDB) tray 800, which can be formed based on the digital model of the patient's teeth with digital brackets disposed thereon. The IDB tray 800 can be used to transfer orthodontic brackets to planned positions, based on the digital model, to the surfaces of the patient's teeth. In some variants, the IDB tray 800 can be used to transfer other orthodontic appliances to the patient's teeth, such as bite turbos, power arms, buttons, archforms, and/or others.

The IDB tray 800 can include cavities 802 configured to receive the teeth of the patient such that the IDB tray 800 can be disposed over the teeth of the patient. The IDB tray 800 can include handles to facilitate handling. The IDB tray 800 can be configured to be placed over an entire dental arch of the patient or a segment thereof. The IDB tray 800 can include wells 804 (e.g., pockets, recesses) sized and shaped to respectively receive orthodontic brackets therein with the contact or bonding surface exposed. The wells 804 can be disposed in the surfaces of the IDB tray 800 forming the cavities 802. The IDB tray 800 can be formed by 3D printing, machining, and/or molding a material over a physical model of the patient's teeth, which may include nonfunctional brackets or protuberances to form the wells 804. The IDB tray 800 can correspond to an entire dental arch of a patient or a portion of the dental arch of the patient. The IDB tray 800 can be distributed into separate segments (e.g., two, three, four, etc.). In some variants, the IDB tray 800 can be broken up into separate segments (e.g., two, three, four, etc.). In some variants, the IDB tray 800 can include a single cavity corresponding to a single tooth of the patient.

In use, the wells 804 of the IDB tray 800 can be loaded with orthodontic brackets based on the digital model. The IDB tray 800 can include instructional and/or identifying information (e.g., a tooth identifier) to assist an operator or clinician in placing the correct type of bracket in the corresponding well 804 accordingly to the plan of the digital model. The contact or bonding surfaces of the orthodontic brackets can be exposed with the brackets disposed in the wells 804.

With the IDB tray 800 loaded up with orthodontic brackets, a clinician or operator can apply an adhesive to the contact surfaces of the orthodontic brackets. The clinician can use the instruction guide 600 to determine the amount of adhesive to apply to the contact surfaces of each of the orthodontic brackets to avoid using too much or too little adhesive. With the IDB tray 800 loaded up with orthodontic brackets with adhesive applied, the IDB tray 800 can be placed over the teeth of the patient, placing the orthodontic brackets at the planned positions corresponding to the digital brackets on the digital model. The adhesive can cure, bonding the orthodontic brackets to the patient's teeth at the planned positions. The adhesive can be cured with air, heat, low temperatures, and/or light, such as UV light. The archform can be deflected from the custom nonplanar shape and coupled to the orthodontic brackets such that the deflected archform exerts forces on the teeth, causing the teeth to move towards the second positions planned in the digital model.

In some variants, the IDB tray 800 can hold an archform therein. The archform can be deflected from the custom nonplanar shape when held within the IDB tray 800 and, upon placement of the orthodontic brackets in the wells 804, be coupled to the orthodontic brackets such that the orthodontic brackets can be bonded to the patient's teeth with the archform. In some variants, the brackets can be bonded to the tooth and the IDB tray 800 can, after bonding, be used to couple the archform to the orthodontic brackets. In some variants, the IDB tray 800 is soluble in water and/or another fluid, enabling the IDB tray 800 to be dissolved after placing the orthodontic brackets and/or archform on the patient's teeth. In some variants, the IDB tray 800 can transfer orthodontic brackets and an archform to the teeth of the patient at the same time.

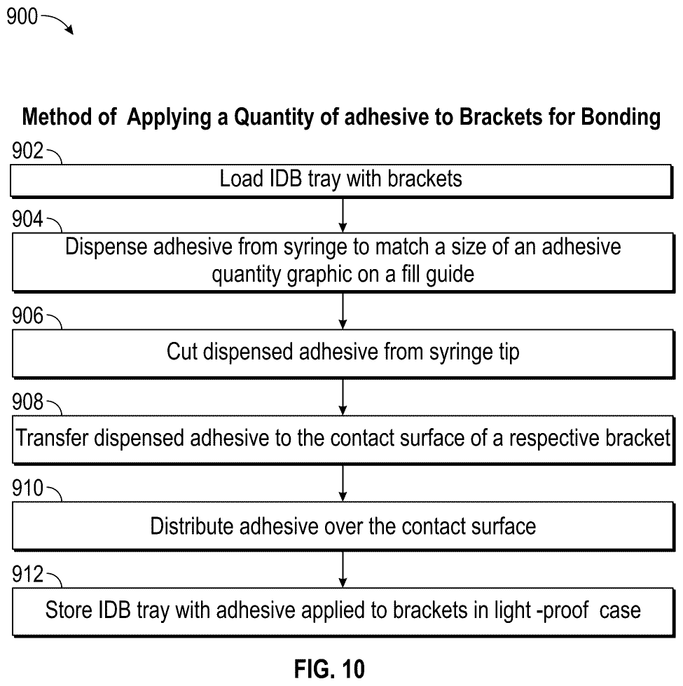

FIG. 10 illustrates a method 900 of applying a quantity of adhesive to orthodontic brackets to bond at locations on surfaces of a patient's teeth. This flow diagram is provided for the purpose of facilitating description of aspects of some embodiments. The diagram does not attempt to illustrate all aspects of the disclosure and should not be considered limiting.

At block 902, an IDB tray can be loaded with orthodontic brackets. As described herein, the selected orthodontic brackets can be placed in wells of the IDB tray based on the digital model.

At block 904, adhesive can be dispensed from a syringe, container, squeeze container, tube, squeeze tube, and/or other device such that a side or horizontal profile of the dispensed adhesive matches a size of an adhesive quantity graphic 610 corresponding to a tooth identifier 608 on the instruction guide 600. As described herein, an opening of the syringe or other device can be the same as the width of the adhesive quantity graphic 610 such that the length (e.g., longitudinal length) of the profile of the dispensed adhesive can be compared with the length (e.g., longitudinal length) of the adhesive quantity graphic 610 to verify that a suitable quantity of adhesive has been dispensed for a specific bracket.

At block 906, the dispensed adhesive can be separated from the syringe or other device. In some variants, a thin, composite instrument can be used to cleanly cut the dispensed adhesive at a tip of the syringe or other device.

At block 908, the dispensed adhesive can be transferred to the contact surface of the orthodontic bracket loaded in the IDB tray 800 that is to be placed on a tooth corresponding to the tooth identifier 608 referenced at block 904.

At block 910, the dispensed adhesive can be distributed over the contact surface of the bracket. In some variants, a microbrush can be used to distribute or pad the dispensed adhesive over the contact surface of the bracket for even coverage. The processed described in reference to blocks 904 through 910 can be repeated until the plurality of orthodontic brackets loaded in the IDB tray 800 have adhesive applied therein and are ready for bonding.

At block 912, the IDB tray 800 loaded with orthodontic brackets with adhesive can, optionally, be stored in a light-proof or airtight case that can prevent the curing or at least slow the curing of the adhesive. This can enable an IDB tray 800 to be prepared for bonding before the arrival of a patient, which can decrease the duration of a patient's appointment. In some variants, this can enable the IDB tray 800 to be shipped to a patient or clinic with brackets prepasted with adhesive for more efficient treatment of patients. As described herein, the loaded IDB tray 800 can be positioned over the teeth of the patient to bond the orthodontic brackets at locations. The adhesive can be cured as described herein, and an archform can be deflected and coupled to the bonded brackets to apply forces to the patient's teeth, moving the patient's teeth toward second positions planned in the digital model. As described herein, in some variants, an IDB tray 800 can be used to transfer the archform to couple to the patient's teeth. In some variants, the IDB tray 800 can transfer the archform and brackets to the patient's teeth at the same time.

Reference is made herein to orthodontic appliances and brackets that move teeth using non-sliding mechanics. However, this disclosure should not be limited to non-sliding mechanics. The methods, apparatuses, and/or systems disclosed herein can be applicable to configurations using sliding mechanics (e.g., an archwire that slides relative to brackets). The methods, apparatuses, and/or systems disclosed herein can be applicable to at least any orthodontic treatment plan that involves coupling an archform to the teeth of the patient and/or bonding and/or placing orthodontic brackets on the teeth of the patient. For example, orthodontic brackets formed and cured from an adhesive, as described herein, can be bonded to a patient's teeth and an archform can be coupled thereto that is configured to slide relative to the cured orthodontic bracket to move teeth of the patient. In some variants, bite turbos, power arms, hooks, and/or other features can be formed, cured, and bonded to the patient's teeth according to a treatment plan.

It is intended that the scope of this present invention herein disclosed should not be limited by the particular disclosed embodiments described above. This invention is susceptible to various modifications and alternative forms, and specific examples have been shown in the drawings and are herein described in detail. This invention is not limited to the detailed forms or methods disclosed, but rather covers all equivalents, modifications, and alternatives falling within the scope and spirit of the various embodiments described and the appended claims. Various features of the orthodontic brackets and archforms described herein can be combined to form further embodiments, which are part of this disclosure. The orthodontic brackets described herein can be bonded to a patient's teeth and the archforms described herein can be deflected and coupled thereto as part of a treatment plan. The archforms can move toward a default position and move the patient's teeth from a first position to a second position. The archforms described herein can be installed in sequence to move the patient's teeth. The orthodontic brackets described herein can be bonded to the teeth of the patient in various orientations, which can include orienting the orthodontic bracket in a first gingival-occlusal orientation and reorienting the orthodontic bracket one hundred and eighty degrees to a second gingival-occlusal orientation (e.g., rotating the orthodontic bracket one hundred and eighty degrees).

Methods of using the orthodontic brackets and/or archforms (including device(s), apparatus(es), assembly(ies), structure(s) or the like) are included herein; the methods of use can include using or assembling any one or more of the features disclosed herein to achieve functions and/or features of the system(s) as discussed in this disclosure. Methods of manufacturing the foregoing system(s) are included; the methods of manufacture can include providing, making, connecting, assembling, and/or installing any one or more of the features of the system(s) disclosed herein to achieve functions and/or features of the system(s) as discussed in this disclosure.

Various other modifications, adaptations, and alternative designs are of course possible in light of the above teachings. Therefore, it should be understood at this time that within the scope of the appended claims the invention may be practiced otherwise than as specifically described herein. It is contemplated that various combinations or subcombinations of the specific features and aspects of the embodiments disclosed above may be made and still fall within one or more of the inventions. Further, the disclosure herein of any particular feature, aspect, method, property, characteristic, quality, attribute, element, or the like in connection with an embodiment can be used in all other embodiments set forth herein. Accordingly, it should be understood that various features and aspects of the disclosed embodiments can be combined with or substituted for one another in order to form varying modes of the disclosed inventions. Thus, it is intended that the scope of the present inventions herein disclosed should not be limited by the particular disclosed embodiments described above. Moreover, while the invention is susceptible to various modifications, and alternative forms, specific examples thereof have been shown in the drawings and are herein described in detail. It should be understood, however, that the invention is not to be limited to the particular forms or methods disclosed, but to the contrary, the invention is to cover all modifications, equivalents, and alternatives falling within the spirit and scope of the various embodiments described and the appended claims. Any methods disclosed herein need not be performed in the order recited. The methods disclosed herein include certain actions taken by a practitioner; however, they can also include any third-party instruction of those actions, either expressly or by implication. For example, actions such as “tying a tie onto an orthodontic bracket” includes “instructing the tying of a tie onto an orthodontic bracket.” The ranges disclosed herein also encompass any and all overlap, sub-ranges, and combinations thereof. Language such as “up to,” “at least,” “greater than,” “less than,” “between,” and the like includes the number recited. Numbers preceded by a term such as “approximately”, “about”, and “substantially” as used herein include the recited numbers (e.g., about 10%=10%), and also represent an amount close to the stated amount that still performs a desired function or achieves a desired result. For example, the terms “approximately”, “about”, and “substantially” may refer to an amount that is within less than 10% of, within less than 5% of, within less than 1% of, within less than 0.1% of, and within less than 0.01% of the stated amount.

1. (canceled)

2. A method of determining a quantity of adhesive to bond an orthodontic bracket to a tooth surface, the method comprising:
receiving scan data corresponding to a scan of an inside of a mouth of a patient;
creating a digital model of teeth of the patient with the scan data;
selecting a digital bracket comprising a digital bonding surface;
positioning the digital bracket at a digital location at a digital surface of a digital tooth of the digital model; and
determining a quantity of adhesive to bond a physical bonding surface of a physical orthodontic bracket, corresponding to the digital bonding surface of the digital bracket, to a physical location of a physical surface of a physical tooth, corresponding to the digital location of the digital surface of the digital tooth, based at least partially on a geometry of the digital bonding surface of the digital bracket and a geometry at the digital location of the digital surface of the digital tooth where the digital bracket is positioned.

3. The method of claim 2, further comprising scanning the inside of the mouth of the patient.

4. The method of claim 3, wherein scanning the inside of the mouth of the patient is performed using a mobile device.

5. The method of claim 3, wherein scanning the inside of the mouth of the patient comprises 3D scanning.

6. The method of claim 2, wherein receiving the scan data comprises receiving the scan data from a mobile device.

7. The method of claim 2, wherein selecting the digital bracket comprises automatically selecting the digital bracket based on one or more characteristics of the digital tooth.

8. The method of claim 2, wherein selecting the digital bracket comprises selecting the digital bracket from a plurality of suggested digital brackets.

9. The method of claim 2, further comprising storing in a memory of a computing device the geometry of the physical bonding surface of the physical orthodontic bracket.

10. The method of claim 2, wherein determining the quantity of adhesive comprises using an algorithm.

11. The method of claim 2, further comprising creating an adhesive quantity graphic to visually indicate at scale the determined quantity of adhesive.

12. The method of claim 11, wherein creating the adhesive quantity graphic comprises creating a profile with a width that matches a width of an opening of a container configured to hold the adhesive and a length corresponding to the determined quantity of adhesive.

13. The method of claim 12, further comprising printing the adhesive quantity graphic to an instruction guide.

14. The method of claim 12, wherein the container is a syringe.

15. The method of claim 2, wherein the physical surface is a lingual surface.

16. A method of determining a quantity of adhesive to bond an orthodontic bracket to a tooth surface, the method comprising:
receiving scan data;
creating with the scan data a digital tooth representative of a corresponding physical tooth of a patient;
positioning a digital bonding surface of a digital bracket at a digital location at a digital surface of the digital tooth; and
determining a quantity of adhesive to bond a physical bonding surface of a physical orthodontic bracket, corresponding to the digital bonding surface of the digital bracket, to a physical location of a physical surface of the physical tooth, corresponding to the digital location of the digital surface of the digital tooth, based at least partially on a geometry of the digital bonding surface of the digital bracket and a geometry at the digital location of the digital surface of the digital tooth where the digital bracket is positioned.

17. The method of claim 16, further comprising storing in a memory of a computing device the geometry of the physical bonding surface of the physical orthodontic bracket.

18. The method of claim 16, further comprising creating an adhesive quantity graphic to visually indicate at scale the determined quantity of adhesive.

19. The method of claim 18, wherein creating the adhesive quantity graphic comprises creating a profile with a width that matches a width of an opening of a container configured to hold the adhesive and a length corresponding to the determined quantity of adhesive.

20. The method of claim 19, further comprising printing the adhesive quantity graphic to an instruction guide.

21. The method of claim 20, wherein printing the adhesive quantity graphic to the instruction guide comprises printing the adhesive quantity graphic onto a sheet.
